(19) United States
(12) Patent Application Publication
(10) Pub. No.: US 2004/0175383 A1
(43) Pub. Date: Sep. 9, 2004

(54) Methods and compositions for treatment of otitis media

(76) Inventors: Philip J. Barr, Oakland, CA (US); Philip A. Pemberton, San Mateo, CA (US); Patrick J. Antonelli, Gainesville, FL (US); Gregory S. Schultz, Gainesville, FL (US); David J. Sundin, Antioch, CA (US)

Correspondence Address: MORRISON & FOERSTER LLP, 755 PAGE MILL RD, PALO ALTO, CA, 94304-1018, US

(21) Appl. No.: 10/731,375

(22) Filed: Dec. 8, 2003

Related U.S. Application Data
(60) Provisional application No. 60/431,286, filed on Dec. 6, 2002.
(60) Provisional application No. 60/435,985, filed on Dec. 20, 2002.

(51) Int. Cl. A61K 39/395; A61K 31/56

(52) U.S. Cl. 424/146.1; 514/171; 424/46

(57) ABSTRACT

The invention is directed to the treatment of otitis media by administration of protease inhibitors. In some embodiments, the protease inhibitors are alpha one-antitrypsin and/or ilomastat.

### CROSS-REFERENCE TO RELATED APPLICATIONS

This application claims priority to U.S. application Ser. Nos. 60/431,286 and 60/435,985, filed Dec. 6, 2002 and Dec. 20, 2002, respectively, the disclosures of both of which are incorporated herein by reference in their entireties.

### FIELD OF THE INVENTION

The invention is directed to the treatment of otitis media by administration of protease inhibitors. In some embodiments, the protease inhibitors are alpha one-antitrypsin and/or ilomastat. The invention finds application in the fields of biomedicine, and human and veterinary therapeutics.

### BACKGROUND OF THE INVENTION

Protease inhibitors have been shown to beneficially impact disease progression in a variety of disease states that involve imbalance in protease-protease inhibitor systems. Examples include metastatic cancer, atopic dermatitis, psoriasis, cystic fibrosis, and chronic obstructive pulmonary disease. The efficacy of protease inhibitors has yet to be studied in the treatment of human otitis media. U.S. Pat. Nos. 5,217,951 and 6,174,859 disclose methods of treatment using alpha one-antitrypsin.

### SUMMARY OF THE INVENTION

The invention provide compositions and methods for the treatment of otitis media using protease inhibitors.

In one aspect, the invention provides a method of treating otitis media in an individual (in some embodiments, a mammal) by administering to the individual (in some embodiments, a mammal) an effective amount of alpha one-antitrypsin. In some embodiments, an effective amount of an antibiotic and/or a steroid is also administered. In some embodiments, the alpha one-antitrypsin is administered in a liquid; in some embodiments the alpha one-antitrypsin is administered as a dry powder. In some embodiments, the mammal to be treated has a perforated tympanic membrane, which in some of the methods of the invention may be due to tympanostomy. In some embodiments, the individual to be treated is a human.

In embodiments, the otitis media is a type of otitis media selected from the group consisting of recurrent acute otitis media (RAOM), chronic otitis media with effusion (COME), acute post-tympanostomy otorrhea (APTO), chronic suppurative otitis media (CSOM), and choleastoma. In some of these embodiments, the type of otitis media is APTO or CSOM. In the latter embodiments, the methods of the invention may further comprise administering an effective amount of an antibiotic.

### DESCRIPTION OF THE INVENTION

The present invention relates to methods and compositions for treatment of individuals suffering from otitis media by administering an effective amount of alpha one antitrypsin (AAT) and/or ilomastat. In some embodiments, AAT alone is administered; in other embodiments, ilomastat alone is administered; in still other embodiments, both AAT and ilomastat are administered in conjunction. AAT is a serine protease inhibitor. Ilomastat is an inhibitor of matrix metalloproteases. In some embodiments, the individual to be treated has a perforated tympanic membrane. In some of these embodiments, the perforated tympanic membrane is due to tympanostomy. In some embodiments, the individual to be treated suffers from a type of otitis media that is recurrent acute otitis media (RAOM), chronic otitis media with effusion (COME), acute post-tympanostomy otorrhea (APTO), chronic suppurative otitis media (CSOM), or choleastoma. In some embodiments, the individual to be treated suffers from otitis media that is acute post-tympanostomy otorrhea (APTO), chronic suppurative otitis media (CSOM), or choleastoma. In some embodiments, the individual to be treated suffers from acute post-tympanostomy otorrhea (APTO) or chronic suppurative otitis media (CSOM). In some embodiments of the invention, the treatment of the individual to be treated is determined based on the bacterial profile of the otitis media.

In some aspects the invention encompasses methods of inhibiting protease activity in an individual suffering from otitis media, or from any of the forms of otitis media or bacterially-caused otitis media described above, by administration to the individual of an effective amount of AAT and/or ilomastat.

Advantages of the present invention include specificity of the agents administered for a variety of proteases known to be present in forms of otitis media, and lack of toxicity of AAT and ilomastat when applied topically, allowing direct application to the site of infection.

Definitions

An “individual” is a vertebrate, preferably a mammal, more preferably a human. Mammals include, but are not limited to, farm animals, sport animals, pets, primates, horses, dogs, cats, mice and rats.

An “effective amount” of drug, compound, or pharmaceutical composition is an amount sufficient to effect beneficial or desired results including modulation of clinical manifestations or symptoms such as a decrease in otomicroscopic findings such as inflammation, erythema, edema, pruritus, or changes in general clinical results such as ear tenderness, otalgia, results of audiograms and other measures of auditory function, fever, loss of appetite, vomiting, tinnitus, dizziness, and odor from the ear, resolution of otorrhea, eradication of pathogen, and decreased relapse rates; or increasing the quality of life of those suffering from the disease (for example, increasing physical functioning, decreasing bodily pain, increasing general health, increasing vitality, increasing social functioning), decreasing the dose of other medications, e.g. palliative care medications or other medications, required to treat the disease, delaying the progression of the disease, decreasing time required for resolution of infection and/or symptoms, and/or prolonging survival of patients. An effective amount can be administered in one or more administrations. For purposes of this invention, an effective amount of drug, compound, or pharmaceutical composition may be an amount sufficient to decrease clinical manifestations of otitis media.

As used herein, two or more agents that are administered “in conjunction” may be administered at the same time or at different times, or in a schedule wherein one or both is administered in multiple doses wherein none of the doses of the agents coincide or one or more of the doses of the agent coincide. Agents administered in conjunction may be administered in the same pharmaceutical vehicle or in separate vehicles, and by the same route or by different routes.

As used herein, “treatment” or “treating” is an approach for obtaining beneficial or desired clinical results such as those listed for “effective treatment.”

### Methods of the invention

With respect to all methods described herein, reference to AAT and/or ilomastat also include formulations comprising one or more of these agents, and formulations comprising other agents in addition to AAT and/or ilomastat. These formulations may further comprise suitable excipients, such as pharmaceutically acceptable excipients including buffers, which are well known in the art. The present invention can be used alone or in combination with other conventional methods of treatment.

Individual to be treated

The individual to be treated by the methods of the invention suffers from or is at risk for otitis media. Methods of diagnosis of otitis media and clinical characteristics of the disease are known in the art. Thus, in some embodiments, the invention includes treatment methods whereby the individual to be treated is selected for treatment based on a diagnosis of otitis media (and in some embodiments, a diagnosis of one or more types of otitis media).

In one aspect, the invention encompasses methods to treat individuals suffering from otitis media wherein there is perforation of the tympanic membrane (TM). Such perforation may be surgically created, or it may occur during the natural course of the disease. In some embodiments the methods of the invention are used in individuals in whom a post-tympanostomy tube has been inserted. The methods may be used as treatment or prophylactically in such embodiments. The methods of the invention reduce the risk, severity, and/or increase the time to possible consequences of tube insertion, including post-tympanostomy tube otorrhea and/or the necessity of tube replacement. For these embodiments, an individual may be selected for treatment based on assessment of the tympanic membrane for perforation (whether arising from deliberate or non-deliberate means).

In one aspect, the invention encompasses methods to treat individuals suffering from a type of otitis media, selected from the group consisting of recurrent acute otitis media (RAOM), chronic otitis media with effusion (COME), acute post-tympanostomy otorrhea (APTO), chronic suppurative otitis media (CSOM), or choleastoma. The individual can have one or more of these types of otitis media. In some embodiments, the individual to be treated suffers from otitis media that is APTO, CSOM, or choleastoma. In other embodiments the individual to be treated suffers from APTO or CSOM. In one embodiment the individual suffers from CSOM.

“Acute otitis media” (AOM), as used herein, refers to a condition characterized by fluid in the middle ear accompanied by signs or symptoms of ear infection (bulging eardrum usually accompanied by pain; or perforated eardrum, often with drainage of purulent or infectious material). A patient with recurrent acute otitis media (RAOM) has had either more than three acute episodes in a period of six months or four or more acute episodes in a period of 12 months.

“Otitis media with effusion” (OME), as used herein, refers to a condition characterized by fluid in the middle ear without signs or symptoms of ear infection. Otitis media with effusion is defined as chronic (COME) when middle ear effusion has been present for at least 3 months.

“Chronic suppurative otitis media” (CSOM), as used herein, differs from “chronic otitis media with effusion” (COME) with respect to the state of the tympanic membrane. Chronic otitis media with effusion (COME) may be defined as a middle ear effusion, without perforation of the tympanic membrane, which is reported to persist for 3 months. Chronic suppurative otitis media is a perforated tympanic membrane with persistent drainage from the middle ear.

“Acute post-tympanostomy otorrhea” (APTO), as used herein, refers to a condition characterized by the presence of purulent fluid or inflamed middle ear mucosa occurs following tympanostomy tubes placement. Drainage following tube placement that persists for less than 8 weeks, is classified as acute.

“Cholesteatomas,” as used herein, are epidermal inclusion cysts of the middle ear or mastoid. They contain the desquamated debris (principally keratin) from their keratinizing, squamous epithelial lining. In the case of a retraction pocket cholesteatoma, the “cyst” opens into the external auditory canal.

In another aspect of the invention, the individual to be treated suffers from infectious otitis media wherein the infective agent comprises one or more species of bacteria and the type of treatment is chosen based on the bacterial profile. Both Streptococcus pneumoniae and Pseudomonas aeruginosa are known to play a role in otitis media, in that acute otitis media is associated with Streptococcus pneumoniae in 39% of the cases whereas chronic otitis media is associated mainly with species of Pseudomonas and Staphylococcus. In some embodiments the treatment regimen may be modified based on the profile of bacteria found. The serine protease HtrA has been shown to play a role in the virulence of Streptococcus pneumoniae, and Pseudomonas aeruginosa secretes a metalloprotease that degrades AAT. Hence the invention includes modification of the choice of protease inhibitor as well as dosage and duration depending on the bacterial profile found in the individual to be treated; e.g., an individual suffering from infection with Pseudomonas aeruginosa may benefit from treatment with both AAT and ilomastat. Methods of determining the presence of these bacterial species are known in the art and bacterial cultures are routinely performed by those of skill in the art. In patients with chronic otitis media, cultures may be obtained to determine the pathogen involved as well as the sensitivity pattern to different classes of antibiotics (particularly when a patient has previously failed a course of therapy).

In some embodiments, the individual to be treated is a mammal, e.g., a human. In some embodiments, the individual is a dog, cat, or horse.

In some embodiments, an individual (such as a mammal, for example, a human) is at risk for developing otitis media. Indicators of risk are known in the art and include clinical history. In these embodiments, administration of AAT and/or ilomastat delays development, ameliorates disease upon or during onset, and/or shortens duration of one or more symptoms. In some embodiments, the individual does not develop symptoms.

Formulations for treatment

The methods of treatment of the invention involve administration of an effective amount of alpha one-antitrypsin (AAT) and/or ilomastat to the individual to be treated.

Human and bacterial proteases play a role in the pathogenesis of otitis media (OM). Proteases are produced by both bacteria and white blood cells. The former may be virulence factors, critical to the establishment of an infection within a host. Leukocyte-derived proteases help to prevent or eradicate bacterial infection, but they may contribute to tissue damage in OM, causing sequelae or disease persistence.

Each class of proteases has its own class of protease inhibitors. Thus, there are serine protease inhibitors, metalloprotease inhibitors, cysteine protease inhibitors, and aspartate protease inhibitors. All known naturally occurring protease inhibitors are proteins, except for some secreted by microorganisms. As with the proteases themselves, the inhibitors contain highly conserved regions and often have a great deal of homology from member to member within a class.

The most well studied proteases and their inhibitors that are involved in OM are those of the metalloprotease and serine protease families. Matrix metalloproteinases (MMPs) and human neutrophil elastase (HNE) are the predominant agents from each family, respectively. Gastric enzymes may also contribute to the pathogenesis of OM via gastropharyngeal reflux.

The serine protease inhibitors include canonical inhibitors, non-canonical inhibitors, and serpins (see, for example, Otlewski, J., Krowarsch, D., and Apostoluk, W., Protein inhibitors of serine proteases, Acta Biochim Polonica, 46:531-565, 1999). Canonical inhibitors bind to the protease in the substrate binding site, and their mechanism of inhibition resembles that of an ideal substrate. Non-canonical inhibitors contain an inhibitory N-terminus which binds to the protease forming a parallel β-pleated sheet. Serpins, the major protease inhibitors in plasma, bind in a manner similar to canonical inhibitors, but their mechanism of action involves the cleavage of a single peptide bond. The serpins are a superfamily of inhibitors, consisting of a single chain with a conserved domain of 370-390 residues (see Potemka, J., Korzus, E, and Travis, J., The serpin superfamily of proteinase inhibitors: structure function, and regulation, J. Biol. Chem. 269:15957-15960, 1994).

AAT is a serine protease inhibitor. AAT has been studied extensively, and the amino acid sequence of the protein was reported by Carrell et al. (Nature 298: 329-334, 1982). The protein has been produced by recombinant methods in yeast; see, e.g., Brake et al., U.S. Pat. No. 4,752,576, Travis et al. (1985) J. Biol. Chem. 260:4384-4389, and published PCT application WO 02/50287. Recombinant AAT, which may be used in the invention, has been used in clinical studies of treatment of individuals with AAT deficiency; see, e.g., Hubbard et al. (1989) J. Clin. Invest. 84:1349-1354. AAT obtained from conventional sources (e.g., human plasma) may also be used in the invention, and is available under the tradename PROLASTIN (Bayer). The major physiological protease targets of AAT include neutrophil elastase, cathepsin G, mast cell chymase, and kallikrein.

Functionally active portions of AAT and other protease inhibitors are known in the art and may be used in the methods of the invention. Further, assays for assessing activity of functionally active portions (whether alone or in the context of a larger sequence) are known. It will be readily understood by those of skill in the art that the native sequence is not necessarily required for a protein to be ftunctionally active. For example, a portion of the protein may be used which retains the desired functionality; this is generally a domain or domains of the protein which are capable of inhibiting one or more proteases. Any such sequence may be used, and any additional sequence may be provided, as long as there is requisite functionality. The functionality need not be as high as the native protein, and thus in some instances may be reduced, the same, or even enhanced as compared to the native protein.

In addition, it is well-understood in the art that amino acid changes, including substitutions, deletions, insertions, post-translational modifications, and the use of amino acid analogs, may be made in the native protein or a portion of the native protein without abolishing or significantly reducing the biological or immunological activity of the protein. Single amino acids may be substituted for others with the same charge or hydrophobicity. Other amino acids may be substituted with amino acids of differing charge or hydrophobicity without significantly altering the function of the protein. It is also contemplated to use variants which enhance the function of the protein as compared to native, or wild type, protein. In addition to substitutions, entire portions of the protein may be deleted without abolishing or significantly affecting the basic biological function of the protein, or extra amino acids inserted without abolishing or significantly affecting the function. Such changes are similar to changes that occur by evolution, and the degree of similarity of two proteins which differ in amino acid sequence can be determined by a method of quantitative analysis such as that described by Pearson and Lipman (Pearson, W. R., and Lipman, D. J., Proc. Natl. Acad. Sci. USA 85:2444-2448, 1998), which compares the homology of amino acid sequences as well as the substitutions of amino acids known to occur frequently in evolutionary families of proteins sharing a conserved function.

As mentioned, functionally active portions of a protease inhibitor that is a protein may be used in the methods of the invention. In the present invention, a “functionally active portion” of a protease inhibitor is a protein that inhibits a protease and that has an amino acid sequence either identical to, or differing in at least one amino acid from, the native form of the protein or a portion of the native form. If the amino acid sequence is different from the native form, the functionally active portion nonetheless has greater similarity to the native sequence or a portion thereof, for example, as defined by the above comparison algorithm of Pearson and Lipman, or other such comparison accepted in the art, than to the amino acid sequence of any other natural polypeptide from the same species. A functionally active portion of AAT is a polypeptide that inhibits neutrophil elastase, cathepsin G, and/or kallikrein, and which has an amino acid sequence which is either identical to the native AAT sequence or a portion thereof or which is more similar to the native AAT sequence or a portion thereof than it is to any other native human protein, for example, as calculated by the algorithm of Pearson and Lipman. Functionally active portions of AAT that may be used in the present invention include, for example, those described in U.S. Pat. Nos. 6,068,994 and 4,732,973, and in A. Hercz, Proteolytic cleavages in alpha-one antitrypsin and microheterogeneity, Biochem. Biophys. Res. Comm. 128: 199-203, 1985. Human AAT is the preferred form for the invention, and the native amino acid sequence is the most preferred form. However, sequences from other species may be used.

Of the metalloproteases, the matrix metalloproteases (MMPs) have been found to be particularly important in a number of normal and pathological conditions. The MMPs, which comprise the collagenases, gelatinases, and stromelysin, have similar structures, with a propeptide, an amino terminal domain, a fibronectin-like domain, a zinc-binding domain, and a C-terminal domain. In addition, some members incorporate a transmembrane domain and a α2V collagen-like domain.

Ilomastat is a highly potent synthetic inhibitor of MMP's that comprises a modified dipeptide analog with the structure N-[2(R)-2(hydroxyamidocarbonylmethyl)-4-methylpentanoyl]-L-tryptophan methyl amide. See, e.g., Grobelny et al. (1992) Biochemistry 31:7152-4, Levy, et al. (1998) J. Med. Chem. 41:199-223.and Galardy, R. E. (1993) Drugs of the Future 18:1109-1111. Ilomastat is available from, e.g., AMS Scientific Inc. PO Box 273269 Concord Calif., 94527, and is manufactured under the trade name GALARDIN; it is also available from CalBiochem.

A major factor to be considered in the use of topical and/or systemic drugs for treatment of otitis media is ototoxicity; i.e., the tendency of certain substances to cause functional impairment and cellular damage to tissues of the external, middle, and especially the inner ear. Unexpectedly, both AAT and ilomastat have been shown in the chinchilla model to lack ototoxicity (see Examples).

The AAT and/or ilomastat may be prepared in any suitable formulation for administration to the individual. Appropriate preparations for various routes of administration are well-known in the art, see, e.g., Remington, The Science and Practice of Pharmacy 20th Ed. Mack Publishing (2000). Topical administration is a useful route for administration and formulations for topical administration are known in the art. In the case of individuals with perforated TM, topical administration can achieve very good delivery; see, e.g., Ohyama et al. (1999) Arch Otolaryngol Head Neck Surg 125:337-340. Powders may be used for formulation in some embodiments of the methods of the invention for use in dry-powder insufflation; see, e.g., Roland (2002) Ear Nose and Throat J. 81 (Suppl. 1): 8-10. A dry powder, e.g., lyophilized, preparation of AAT and/or ilomastat, with or without excipients, may be employed. Eardrops are also commonly used to deliver various agents in CSOM and other types of otitis media, such as those used for neomycin/polymyxin B/hydrocortisone otic suspension; such drops may also be used for delivery of AAT and/or ilomastat. An earspray may also be used for delivery by mechanical pump or by aerosolization; droplets in the range of from about 5, about 10, about 20, or about 50 microns to about 50, about 100, about 150, or about 300 microns are useful in such an earspray. An ear catheter can also be used to deliver formulations to the middle ear. Slow release agents, as are known in the art, may also be employed, e.g., AAT and/or ilomastat embedded in a biodegradable gel, pellet, tablet, or capsule.

If AAT and ilomastat are to be used in conjunction, they may be prepared in the same formulation or in separate formulations. Similarly, if another therapeutic or palliative agent is to be used with either or both of AAT and/or ilomastat, it may be prepared in the same or different formulation.

Within the scope of the invention described herein is the use of pharmaceutical formulations containing a combination of the protease inhibitor(s) and one or more additional pharmaceutically active agents. Pharmaceutically active agents useful in the invention include, without limitation, antibiotics, antifungals, antiviral agents, local anesthetics, anti-inflammatory drugs (e.g., salicylates, colchicine, para-aminophenol, propionic acid, piroxicam, ketorolac, ketoprofen, cyclooxygenase type II inhibitors and indomethacin, among others), corticosteroids, pH altering agents that make the environment more acidic and less friendly to bacteria, drying agents to reduce moisture in the ear and make it less hospitable to pathogens, ceruminolytic agents, and agents (e.g., antihistamines or scopolamine) that are used to treat vestibular dysfunction of the inner ear (e.g., vertigo, disequilibrium).

Corticosteroids include, for example, hydroxytriamcinolone, alpha methyl dexamethasone, dexamethasone acetate, betamethasone, beclomethsasone dipropionate, betamethasone benzoate, betamethasone dipropionate, betamethasone valerate, clobetasol valerate, clobetasol propionate, desonide, desoxymethasone, dexamethasone, difluorosone diacetate, diflucortolone valerate, fluadrenolone, fluclorolone acetonide, flumethasone pivalate, fluocinolone acetonide, fluocinonide, flucortine butylester, flucortolone, fluprednidine (fluprednylidene) acetate, flurandrenolone, halcinonide, hydrocortisone acetate, hydrocortisone butyrate, hydrocortisone valerate, 11-desoxycortisol, methylprednisolone, triamcinolone, triamcinolone acetonide, triamcinolone diacetate, triamcinolone hexacetonide, cortisone, cortodoxone, flucetonide, fludrocortisone, difluorosone diacetate, fluradrenolene acetonide, medrysone, amcinafel, amcinafide, betamethasone and the balance of its esters, chloroprednisone, clocortelone, clocortelone pivalate, clescinolone, dichlorisone, difluprednate, flucloronide, flunisolide, fluoromethalone, fluperolone, fluprednisolone, hydrocortisone, meprednisone, paramethasone, paramethasone acetate, prednisolone, prednisolone acetate, prednisolone tebutate, prednisone, beclomethasone dipropionate, alclometasone dipropionate, mometasone furoate, or combinations thereof.

Antibiotics include macrolide antibiotics, penicillins, tetracyclins, cephalosporins, quinolones, fluoroquinolones, neomycin, gentamycin, vancomycin, or a combination thereof.

Clinically, macrolide antibiotics are used principally for treating infections with Streptococci, Staphylococci, and Pneumococci. Generally the toxicity of macrolide antibiotics is low. Esters of macrolide antibiotics have become therapeutically important because they result rapidly in higher blood levels, and further they are practically free of odor and are highly stable. Macrolide antibiotics are classified according to the size of the macrocyclic lactone ring. Macrolide antibiotics are polyfunctional molecules, most of which have at least one amine sugar and are basic.

Suitable macrolide antibiotics include those with 12-member lactone rings such as methymycin and neomethymycin. Also included are macrolide antibiotics with 14-member lactone rings, of which the preferred representatives are the erythromycins, produced from Streptomyces erythreus. Examples include, erythromycin A, erythromycin B, erythromycin C, erythromycin D, erythromycin E, erythromycin estolate, erythronolid, and clarythromycin. Other examples of macrolide antibiotics with 14-member lactone rings include, megalomycin and its derivatives, picromycin, narbomycin, oleandomycin, triacetyl-oleandomycin; and the neutral compounds laukamycin, kujimycin A, albocyclin, and cineromycin B.

Macrolide antibiotics having 16-member rings include, carbomycin (Magnamycin) and its derivatives (i.e. niddamycin), spiramycin and its derivatives, leucomycin and its derivatives (i.e. midecamycin, maridomycin, tylosin, cirramycin, and juvenimicins); and the neutral representatives chalcomycin and neutramycin. Examples of macrolide antibiotics with larger lactone rings, i.e. having 26-40 or more ring members, include pimaricin, lucensomycin, nystatin, amphotericin B, hamycin, candicidin A and B, candidin, and levorin. The effectiveness of this group is practically exclusively against fungi and yeasts.

Therapeutic formulations of AAT and/or ilomastat and/or other agents used in accordance with the present invention may be prepared for storage by mixing a protease inhibitor or combination of inhibitors having the desired degree of purity with optional pharmaceutically acceptable carriers, excipients or stabilizers (Remington, The Science and Practice ofPharmacy 20th Ed. Mack Publishing (2000)). In some embodiments, such formulations may be in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid, tocopherol, and methionine; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Sustained-release preparations may be prepared, e.g., in the form of gels for topical application.

Preservatives are optionally included in the formulation used in the invention to maintain the integrity of the formulation. It is known that formulations containing an aqueous phase in combination with a protein are susceptible to attack by bacteria and fungi. Microbial growth not only contaminates the formulation but is potential toxicity hazard and a source of infection for patients. It is especially important to minimize microbial growth in topical formulations applied to broken or inflamed skin. Viscosity degradations reported with some polymers when exposed to microbial contamination is also of concern. Preservatives useful in the formulations include, for example, without limitation, quatemium, methylparaben, phenol, para-hydroxybenzoate compounds, propyleneglycol, propylparaben, or a combination thereof. Other useful preservatives include octadecyldimethylbenzyl ammonium chloride; hexamethonium chloride; benzalkonium chloride, benzethonium chloride; butyl or benzyl alcohol; catechol; resorcinol; cyclohexanol; 3-pentanol.

The formulations to be used for in vivo administration are preferably sterile. This is readily accomplished by, for example, filtration through sterile filtration membranes.

The formulations used in the methods of the present invention may be in unit dosage forms such as powders, solutions, gel-based dosage units, or suspensions, for administration by topical or insufflation routes.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the otic, oral or nasal respiratory route for local or systemic effect. Solution, suspension or powder compositions may be administered otically, orally or nasally, from devices which deliver the formulation in an appropriate manner. Further routes of delivery may be found in the art, e.g., Ohyama et al. (1999) Arch Otolaryngol Head Neck Surg 125:337-340.

Administration of AAT and/or Ilomastat and Assessment of Treatment

The AAT and/or ilomastat may be administered to an individual via any suitable route. Topical delivery and dry powder insufflation are especially effective in the case of perforated TM, as noted above. However, any route that provides an effective dose to the site of otitis media may be used, as apparent to one of skill in the art. It should be apparent to a person skilled in the art that the examples described herein are not intended to be limiting but to be illustrative of the techniques available. In some embodiments the AAT and/or ilomastat may be administered by more than one route, e.g., topically and systemically. Liquid formulations may be delivered as ear drops or an ear spray, or may be delivered via an ear catheter, as is known in the art. Ear sprays may be delivered by mechanical pump or via aerosolization. Depending on the route of administration, commercially available nebulizers for liquid formulations, including jet nebulizers and ultrasonic nebulizers may be useful. Liquid formulations can be directly nebulized and lyophilized powder can be nebulized after reconstitution. Alternatively, aerosolized formulations may be use in some forms of administration, using a fluorocarbon formulation and a metered dose dispenser, or as a lyophilized and milled powder.

The particular dosage regimen, i.e., dose, timing and repetition, will depend on the particular individual and that individual's medical history. A single dose or repeated doses may be given of one or more agents described herein. For repeated administrations over several days or longer, depending on the condition, the treatment is sustained until a desired suppression of disease symptoms occurs or until sufficient therapeutic levels are achieved to reduce the risk of, for example, the necessity for placement of second tympanostomy tube. The progress of therapy is easily monitored by conventional techniques and assays. The dosing regimen can vary over time.

For the purpose of the present invention, the appropriate dosage of AAT and/or ilomastat will depend on the combination (e.g., one or both of the agents, or compositions thereof) employed, the type and severity of the otitis media to be treated, whether the agent is administered for preventive or therapeutic purposes, previous therapy, the patient's clinical history and response to the agent, and the discretion of the attending physician.

Typically the clinician will administer AAT and/or ilomastat until a dosage is reached that achieves the desired result. A single dose of AAT to be delivered to the middle ear can range from about 0.1 mg, 1 mg, 3 mg, 5 mg, 8 mg, 10 mg, or 20 mg, to about 1 mg, 3 mg, 5 mg, 8 mg, 10 mg, 20 mg, or 50 mg. In some embodiments, a single dose of AAT is from about 0.1 mg to about 50 mg, or from about 1 mg to about 20 mg, or from about 1 mg to about 10 mg, or from about 3 mg to about 8 mg, or about 5 mg. If the AAT is delivered, for example, in the form of a liquid (e.g., by ear drop), an exemplary dose is 100 microliters of a 50 mg/ml solution of AAT in a suitable liquid carrier. Dose frequency may be from once daily, twice daily, or three times daily, to twice daily, three times daily, four times daily, five time daily, or six times daily. In some embodiments, the dose frequency is from once daily to six times daily, or once daily to four times daily, or once or twice daily. Frequency of administration may be determined and adjusted over the course of therapy, and is generally, but not necessarily, based on treatment and/or suppression and/or amelioration and/or delay of symptoms and clinical findings. Alternatively, sustained continuous release formulations of AAT may be appropriate. Thus, dosing schedule is also influenced by the type and route of administration (e.g., sustained release or continuous infusion via ear catheter). Various formulations and devices for achieving sustained release are known in the art. In one embodiment, dosages for AAT may be determined empirically in individuals who have been given one or more administration(s) of AAT based on results of the initial administration(s). The AAT formulation may be administered for a duration of up to one year depending on the indication (e.g., treatment of inflammation associated with otitis media to prophylaxis in patients post tympanostomy tube placement). Higher or lower doses may be used at the discretion of the clinician, as well as greater or lesser frequency of application.

A single dose of ilomastat to be delivered to the middle ear can range from about 0.1 mg, 1 mg, 3 mg, 5 mg, 8 mg, 10 mg, or 20 mg, to about 1 mg, 3 mg, 5 mg, 8 mg, 10 mg, 20 mg, or 50 mg. In some embodiments, a single dose of ilomastat is from about 0.1 mg to about 50 mg, or from about 1 mg to about 20 mg, or from about 1 mg to about 10 mg, or from about 3 mg to about 8 mg, or about 5 mg. If the ilomastat is delivered, for example, in the form of a liquid (e.g., by ear drop), an exemplary dose would be 100 microliters of a 50 mg/ml solution of ilomastat in a suitable liquid carrier. Dose frequency may be from once daily, twice daily, or three times daily, to about twice daily, three times daily, four times daily, five time daily, or six times daily. In some embodiments, the dose frequency is from once daily to six times daily, or once daily to four times daily, or once or twice daily, or once daily or twice daily. Frequency of administration may be determined and adjusted over the course of therapy, and is generally, but not necessarily, based on treatment and/or suppression and/or amelioration and/or delay of symptoms and clinical findings. Alternatively, sustained continuous release formulations of ilomastat may be appropriate. Various formulations and devices for achieving sustained release are known in the art. In one embodiment, dosages for ilomastat may be determined empirically in individuals who have been given one or more administration(s) of ilomastat based on results of the initial administration(s). The ilomastat formulation may be administered for a duration of up to one year depending on the indication (e.g., treatment of inflammation associated with otitis media to prophylaxis in patients post tympanostomy tube placement). Higher or lower doses may be used at the discretion of the clinician, as well as greater or lesser frequency of application.

Administration of AAT and/or ilomastat in accordance with the methods in the present invention can be continuous (e.g., by sustained release formulations) or intermittent, depending, for example, upon the recipient's physiological condition, whether the purpose of the administration is therapeutic or prophylactic, and other factors known to skilled practitioners. The administration of AAT and/or ilomastat may be essentially continuous over a preselected period of time or may be in a series of spaced dose, e.g., either before, during, or after tympanostomy and tube placement, before, during, before and after, during and after, or before, during, and after tympanostomy and tube placement.

In some embodiments, AAT alone is administered, in some embodiments ilomastat alone is administered, and in some embodiments the two protease inhibitors are administered in conjunction. In the latter case, the two may be administered simultaneously, by the same or different routes, in the same or different formulations, at separate times, on the same or separate schedules, or any combination of the preceding. The dose, frequency, and duration for each agent given above may be combined in any combination to produce a therapeutic effect. AAT and/or ilomastat can also be used in conjunction with other agents that serve to enhance and/or complement the effectiveness of the protease inhibitors, as described above.

Indicia of Effectiveness.

Treatment efficacy can be assessed by methods well-known in the art. Indicia of efficacy include clinical manifestations such as reduced ear tenderness, reduced otalgia, stabilized or improved hearing (e.g. as manifested in audiogram results), resolution of otorrhea, eradication of pathogen, reduced odor from the ear, no necessity for surgery or no need for further surgery, and prevention of future development of disease. Quality of life measures may also be used to assess efficacy, such as physical functioning, bodily pain, general health, vitality, and social functioning. When the methods of the invention are used prophylactically or therapeutically in post-tympanostomy situations, an indicia of efficacy is reduction of probability of need for second tube placement, an intact tube, reduction of probability or reduction of severity of posttympanostomy otitis media and/or any of the indicia of efficacy listed previously. Visual inspection of the tympanic membrane may also be used to judge treatment efficacy, e.g., otomicroscopy may be used to assess inflammation, erythema, edema, and pruritus. Bacterial culture may be performed if effusion is present. Other clinical indicia are known to those of skill in the art.

### Kits

The invention also provides kits for use in the treatment of otitis media. Kits may include the compositions of the invention, such as compositions containing alpha one-antitrypsin and/or ilomastat, and, in some embodiments compositions containing antibiotics and/or steroids, in suitable containers, and any materials necessary or useful in the administration and use of the compositions in the methods described above. In some embodiments of the invention the composition(s) is/are provided in a container, and optionally further packaging for segregation from other components of the kit and/or to facilitate dispensing, and a set of instructions for use of the composition(s). The instructions may inform the user of methods for administration of the composition(s) of the invention, suggested dosages and schedules for various forms of otitis media. The instructions may be in any form, and provided, e.g., as a separate insert or on a label that is affixed to the container or packaging. Instructions include instructions for any of the methods described herein. In some embodiments, instructions are directed to the use of alpha one-antitrypsin and/or ilomastat in the treatment of otitis media. In some embodiments, instructions are directed to the use of alpha one-antitrypsin in the treatment of otitis media. In some embodiments, the instructions further are directed to the use of an antibiotic and/or a steroid, which may optionally also be included in the kit, in conjunction with alpha one-antitrypsin and/or ilomastat, for the treatment of otitis media. In some embodiments, instructions are directed to treating a type of otitis media with alpha one-antitrypsin and/or ilomastat, where the type of otitis media is selected from the group consisting of recurrent acute otitis media (RAOM), chronic otitis media with effusion (COME), acute post-tympanostomy otorrhea (APTO), chronic suppurative otitis media (CSOM), and choleastoma. In some embodiments the instructions for treatment of a type of otitis media further comprise instructions for administering an effective amount of an antibiotic In some embodiments, instructions are directed to the treatment of mammals, and in some embodiments the instructions are directed to the treatment of humans.

Exemplary optional additional components of kits of the invention include diluent for compositions to be reconstituted, and components to facilitate the administration of alpha one-antitrypsin, and/or ilomastat, as well as other components of the kit such as antibiotics and/or steroids.

### EXAMPLES

### Example 1

### Protease Levels and Inhibition of Protease with AAT and/or Ilomastat in Human Otitis Media

Human Subjects & Sample Collection: Middle ear effusion (MEE) samples were collected from all consenting patients that presented to the investigators for the treatment of OM. Most samples were taken from subjects at the time of myringotomy, with or without tube placement, for recurrent acute otitis media (RAOM) and chronic otitis media with effusion (COME). Less commonly, samples were collected upon presentation with acute post-tympanostomy otorrhea (APTO) or with chronic suppurative otitis media (CSOM). Samples were aspirated with a Juhn Tymp-Tap (Medtronic-Xomed, Jacksonville, Fla.). The aspiration device was rinsed—and sample diluted—with 500 μl of normal saline and immediately placed on ice for transport to the investigators' laboratories. Samples were centrifuged to remove cellular material then divided into aliquots and frozen until batch processing could be performed.

Protease and Inhibition Analysis: Active (aMMP) and proenzyme (pMMP) forms of MMPs 2 and 9 were measured using a gelatin zymography technique. MMP activity (tMMP, but predominantly MMPs 2 and 9) present in MEE samples was measured using a calorimetric assay that uses a synthetic substrate that reduces Ellmans reagent upon cleavage. Because two different activity assays were used that detected different types and levels of MMPs, a direct comparison between aMMP, pMMP and tMMP was not considered valid. Human neutrophil elastase (HNE) activity was measured using a standard technique. Results were expressed as change in absorbance over time (mAU/min). If insufficient sample was available (e.g., scant or extraordinarily thick effusions), Samples with excessive activity levels were tested by serial dilution. MMP activity was measured in the presence of physiologically deliverable levels of ilomastat. HNE activity was measured in the presence of physiologically deliverable levels of rAAT. rAAT was expressed in recombinant yeast cells essentially as described by Travis et al., J. Biol. Chem. 260:4384;4389 (1985), and purified by column chromatography.

Statistical Analysis: An analysis of variance was performed on the enzyme activities and mean inhibition of activities to determine if there were differences activities across diagnoses, middle ear findings. If there were statistically significant differences, Tukey's LSD tests were performed. Enzyme activity greater than 3 mAU/min and enzyme inhibition of more than 30% were considered clinically significant . The distribution of patients with an activities greater than 3 mAU/min and inhibition greater than 30% was tested using Fisher's exact test to determine if there was a difference in these distributions across diagnoses.

### Results

A total of 100 patients were enrolled in the study, yielding 144 MEEs for analysis. Study subjects were primarily children undergoing tympanostomy tube placement; thus, the study age was heavily biased toward young children (Table 1). Male subjects and mucoid MEEs predominated across all diagnostic groups.

MMP and HNE activities varied dramatically. Significant levels of MMP and HNE (>3 mAU/min) were found in 52% and 37% of MEEs, respectively. Mean total MMP levels were significantly higher (p=0.0032) in APTO than COME, RAOM, COME/RAOM, and chronic mucositis (Table 2). There was no statistically significant difference in the mean activities of aMMP2, aMMP9, and pMMP9 across the diagnoses. For pMMP2 there was a statistically significant difference (p=0.005), with the mean activity for cholesteatoma significantly greater than the means of the other diagnoses. Similarly, there was no statistically significant difference in the mean activities of aMMP2, aMMP9, and pMMP9 as a function of MEE type, but the mean activity of pMMP2 was significantly higher in purulent MEEs (p=0.012). Both of these significant results were highly influenced by a single subject with a pMMP2 value of 392,482. When this subject was deleted from the analysis, there were no significant differences between the diagnoses or findings for pMMP2. Mean HNE activity levels were significantly (p<0.0001) higher in cholesteatoma, chronic mucositis, and post-tube otorrhea than in COME, COME/RAOM, and RAOM (Table 2).

Overall, ilomastat inhibited 64% of MMP activity and rAAT inhibited 75% of HNE activity. Ilomastat and rAAT demonstrated significant inhibition (>30% reduction) in 80% and 82% of MEEs with significant levels of MMP and HNE activity (i.e., >3 mAU/min), respectively (Table 3). There was a statistically significant difference in the mean inhibition of MMP (p=0.001) across the diagnoses. There was no difference in inhibition between COME, COME/RAOM, and RAOM or between cholesteatoma, CSOM, and APTO; however, the latter had higher mean percent inhibition than the former. Analysis of HNE activity indicated there was a significant difference in mean inhibition of activity across the diagnoses (p<0.0063), with COME/RAOM showing significantly less inhibition than the other diagnoses.

The ultimate goal of our investigation was to evaluate the human therapeutic utility of protease inhibitors, rAAT and ilomastat, in OM. Toward that end, we measured MMP and HNE activity effecting the absence and presence of these protease inhibitors. We observed that MMP and HNE activity is commonly present in a wide range of human OM. Not surprisingly, the neutrophil-derived HNE was found in higher levels in suppurative conditions such as cholesteatoma, chronic suppurative otitis media, and acute post-tympanostomy otorrhea. MMP, being derived from both host and bacterial sources, did not vary significantly across the different types of OM.

We observed that MEEs with significant levels of MMP and HNE activity were inhibited by ilomastat and rAAT.

These observations suggest that there is therapeutic potential for their use in human subjects. Ilomastat is a broad spectrum MMP inhibitor that has shown activity in a number of biological systems, including animal wound healing models and human clinical trials for bacterial keratitis. In fused human plasma-derived AAT (Prolastin™, Bayer Corporation) has been shown to be safe and efficacious in the treatment of emphysema that is secondary to AAT deficiency. Similarly, rAAT has also been shown previously to be safe when administered by inhalation to patients with AAT-deficiency. Topically-administered Prolastin™ has also been shown to have beneficial effects in the treatment of human atopic dermatitis, and infused Prolastin™ was also shown to have a favorable, albeit marginal impact in the therapy of neonatal respiratory distress syndrome. Topically-administered rAAT and ilomastat have recently been shown to be non-ototoxic; therefore, consideration should be given to the use of rAAT and ilomastat in clinical trials on the safety and efficacy of these agents for the treatment of otitis media.

TABLE 1
Subject demographics and fluid characteristics

| | | | | Age | | | |
|---|---|---|---|---|---|---|---|
| Diagnosis | Subjects | Ears | Male % | (SD) | Serous % | Mucoid % | Purulent % |
| COME | 54 | 81 | 61 | 4.7 | 31 | 69 | 0 |
| | | | | (3.8) | | | |
| RAOM | 13 | 21 | 69 | 2.5 | 14 | 67 | 19 |
| | | | | (1.1) | | | |
| RAOM/COME | 7 | 10 | 57 | 2.8 | 20 | 80 | 0 |
| | | | | (2.1) | | | |
| APTO | 14 | 16 | 57 | 3.1 | 12 | 19 | 69 |
| | | | | (2.8) | | | |
| CSOM | 6 | 9 | 33 | 43.3 | 22 | 33 | 45 |
| | | | | (38.5) | | | |
| Cholestea | 6 | 7 | 66 | 18.3 | 14 | 29 | 57 |
| toma | | | | (4.5) | | | |

Age is shown in years, and standard deviations are shown in parentheses.

TABLE 2
Enzyme activities by diagnosis

| Diagnosis | pMMP2* | pMMP9* | aMMP2* | aMMP9* | tMMP* | HNE* | MMP^{†} | HNE^{†} |
|---|---|---|---|---|---|---|---|---|
| COME | 3433 | 4623 | 1347 | 8725 | 4.4 | 6.0 | 42 | 20 |
| | (11824) | (29000) | (3697) | (62514) | (5.9) | (17.1) | | |
| RAOM | 461 | 366 | 281 | 840 | 5.9 | 4.2 | 68 | 44 |
| | (791) | (592) | (325) | (1401) | (5.5) | (7.1) | | |
| RAOM/ | 3215 | 148 | 325 | 289 | 3.4 | 5.3 | 33 | 33 |
| COME | (5805) | (106) | (412) | (206) | (3.9) | (8.8) | | |
| APTO | 21410 | 1342 | 429 | 5866 | 11.8 | 49.9 | 75 | 88 |
| | (51491) | (2927) | (134) | (18380) | (12.8) | (31.9) | | |
| CSOM | 8602 | 2999 | 934 | 20176 | 4.2 | 72.4 | 71 | 57 |
| | (13654) | (6015) | (895) | (52399) | (2.9) | (80.0) | | |
| Cholest- | 79711 | 1327 | 379 | 487 | 8.0 | 78.0 | 63 | 71 |
| eatoma | (174848) | (1904) | (375) | (210) | (8.4) | (108.2) | | |

Matrix metalloproteinase (MMP) values are shown for active (a) and proenzyme (p) forms, types (2 & 9) and total (t).
Human neutrophil elastase is represented as HNE.
Standard deviations are shown in parentheses.
*Mean activity (and range) in mAU/minute.
^{†}Percentage of samples with activity > 3 mAU/minute.

TABLE 3
Inhibition of enzyme activity

| | Ilomastat- | MMP* | | HNE* |
|---|---|---|---|---|
| | MMP | >30% | A1AT-HNE | >30% |
| Diagnosis | Inhibition %^{†} | Inhibition | Inhibition %^{†} | Inhibition |
| COME | 70.0 | 86 | 76.9 | 87 |
| | (31.0) | | (34.3) | |
| RAOM | 85.5 | 100 | 60.7 | 63 |
| | (7.8) | | (46.8) | |
| RAOM/ | 81.4 | 100 | 25.7 | 40 |
| COME | (12.1) | | (40.7) | |
| APTO | 41.2 | 57 | 86.2 | 93 |
| | (28.1) | | (23.8) | |
| CSOM | 51.5 | 40 | 99.3 | 100 |
| | (32.7) | | (0.7) | |
| Cholestea | 31.5 | 71 | 94.1 | 100 |
| toma | (32.4) | | (2.7) | |

*Percent of samples that had enzyme activity inhibited by >30%.
^{†}Mean inhibition. Standard deviations are shown in parentheses.

### Example 21

### AAT Reduces Time to Resolution of Experimentally Induced Middle ear Infection in the Chinchilla

The therapeutic benefits of otic administration of rAAT in an acute otis media animal model were assessed in chinchillas. All study animals were initially anesthetized to allow baseline hearing testing

(electrocochleography and tympanometry) and to allow bacteria (Streptococcus pneumoniae) to be injected across the thin bone covering the middle ear (dorsal bulla) to induce a middle ear infection bilaterally. After allowing the inflammation to become established for 3 to 4 days, the ears were graded for severity of middle ear inflammation. Half the ears were injected with alpha 1-antitrypsin (100 mg/ml)half with saline. All animals received systemic antibacterial treatment (enrofloxacin). Eardrums were serially examined otomicroscopically and tympanometrically under anesthesia every 2 days for 16 days. Assessment was given quantiatively according to the following table:

| TM | |
|---|---|
| 0 | NORMAL |
| 1 | GRAY OR WHITE, |
| | OPAQUE |
| 2 | RED, |
| | TRANSLUCENT |
| 3 | RED, |
| | OPAQUE |
| 4 | YELLOW. TRANSLUCENT |
| 5 | YELLOW, |
| | OPAQUE |
| 6 | PERFORATED |

Hearing assessment were also performed throughout the study and following euthanasia at the end of study, temporal bone specimens were obtained from all ears for histopathological analysis.

Results from the study indicated that there were no differences in changes of auditory thresholds in rAAT- and control-treated ears, indicating that there was no significant ototoxicity associated with the rAAT otic administration. More importantly, however, otomicroscopic data, an assessment of middle ear inflammation derived from the clinical appearance of the eardrum; indicated that rAAT mitigated the inflammatory process more rapidly than saline. See Table, below:

TM Score

| | Day | | | | | | | | | | | | | |
|---|---|---|---|---|---|---|---|---|---|---|---|---|---|---|
| | 1 | 4 | 6 | 8 | 10 | 12 | 14 | 16 | 18 | 20 | 22 | 24 | 26 | 28 |
| rAAT | 0 | 2.167 | 4.17 | 3.5 | 2.8 | 2.1 | 1.25 | 0.75 | 0.5 | 0.5 | 0 | 0 | 0 | 0 |
| vehicle | 0 | 2.167 | 4.33 | 3.583 | 2.5 | 2.2 | 1.88 | 2.375 | 1 | 1.5 | 1.75 | 1.5 | 0 | 0 |

This example showed that a single application of rAAT reduced time to resolution of clinical manifestations of otitis media from 26 days to 22 days.

### Example 3

### Protease Inhibitors AAT and Ilomastat are not Toxic in the Chinchilla

The purpose of this study was to assess the safety of protease inhibitors when instilled into the middle ear, with a view to their potential use as human therapeutic agents. Prospective, randomized, controlled trial in the chinchilla model. The chinchilla has been widely used by researchers for studies of ototoxicity.

After completing baseline auditory testing and bilateral transpalatal obstruction of the Eustachian tube (ETO), chinchillas received weekly transbullar injections of protease inhibitor (alpha 1-antitrypsin, ilomastat, or both), vehicle, or saline. After one month, hearing was tested and the animals were sacrificed. Temporal bone histopathology was performed.

All treatment groups demonstrated a statistically insignificant average loss in long-term hearing (0 db) for all measures using clicks and tones (p>0.15 for all conditions). All treatment groups were statistically insignificantly different from one another (p=0.5625). Protease inhibitors that are currently under study in human clinical trials for inflammatory conditions have no significant toxic effect on the inner ear of chinchillas. These findings support the safety of further clinical trials using these inhibitors to treat middle ear inflarmation.

A total of 96 healthy adult chinchillas of either sex, 400-600 grams, were used in this experiment. Prior to entry into the study, all animals were otomicroscopically free of middle ear pathology. All animals underwent auditory testing and bilateral transpalatal obstruction of the Eustachian tube, followed by immediate transbullar injection (across the thin bone covering the dorsal aspect of the middle ear) of saline or vehicle solution, with or without protease inhibitor. Thus, there were five treatment groups: saline, vehicle solution, vehicle with alpha 1-antitrypsin (100 μg /ml), vehicle with ilomastat (100 μg /ml), and vehicle with alpha 1-antitrypsin and ilomastat (100 μg /ml of each). Weekly thereafter, animals received a light anesthetic for ear examinations, transbullar sampling of the middle ear fluid, and transbullar reinjection of saline or the vehicle +/− protease inhibitor. One month after ETO, animals were anesthetized, middle ear fluid was removed, and auditory testing was performed immediately prior to sacrifice. Temporal bones were harvested for histopathological analysis.

Solution Preparation: Bulk ca. 5% alpha 1-antitrypsin was composed of the following ingredients:

| | |
|---|---|
| KCl | 200 mEq/L |
| Sodium Phosphate | 0.02 M |
| Sodium Citrate | 0.005 M |
| N-Acetyl-Cysteine | 0.005 M |
| pH | 7.5 ± 0.2 |
| Recombinant alpha 1-antitrypsin | 51.65 mg/mL |

Otic solution, 1% alpha 1-antitrypsin, was constituted by diluting the bulk ca. 5% solution as follows:

| | |
|---|---|
| Alpha 1-antitrypsin (51.65 mg/mL) | 20 mL |
| Quaternium 15 | 0.02 mL |
| Buffer, pH 7.4, 50 mM KCL | 79.98 mL |

Ilomastat was prepared by substituting alpha 1-antitrypsin. Vehicle was similarly prepared, without the addition of any protease inhibitor. Injectable, 0.9% normal saline was used as the non-treatment control.

Eustachian Tube Obstruction (ETO): Bilateral ETO was performed with a transoral, transpalatal approach as described by Paparella and colleagues. Briefly, the palate was split and the Eustachian tube orifices were bluntly palpated. The orifices were denuded of mucosa, the deeper tissues cauterized, and the lumen packed with Gelfoam sponge. The palate was reapproximated with a single layer of polyglycolic acid sutures.

Auditory Evaluation: Assessment of auditory thresholds was performed using electrocochleography. Needle electrodes were positioned over the bullae (reference), the vertex (active), and the neck (ground). Electrocochleographic thresholds were measured for clicks and tone pips at 4, 8, 12, and 16 kHz. Stimulus generation was executed by an auditory electrophysiology workstation with SigGen™ and AeP™ software (Tucker-Davis Technologies, Gainesville, Fla.) and Etymotic transducers (ER-2, Elk Grove Village, Ill.). Stimuli were introduced with an insert earphone tube placed into the external auditory canal, just medial to the crus of the helix. Auditory thresholds were evaluated by decreasing stimulus intensity in 5 dB increments, from a maximum of 100 dB, until the waveform disappeared. At that point, the stimulus intensity was increased in 5 dB increments until the waveform re-emerged.

Threshold measurements were made after ETO and immediately following the final middle ear aspiration after one month of exposure to the test substances. Any auditory threshold values that exceeded the upper limits of detection (i.e. >100 dB) were given a value of 118 dB.

Middle Ear Sampling Techniques: Middle ear fluid was sampled as previously described. Samples were aspirated through a polyethylene catheter, carefully passed through a 15 gauge needle from the superior to the inferior bulla to avoid trauma to the tympanic membrane. A second 23 gauge needle vented the superior bulla to prevent tympanic membrane perforation during aspiration. The superior bulla was prepared with povidone-iodine prior to middle ear aspiration. Otomicroscopy was repeated after aspiration to document tympanic membrane integrity.

Anesthesia: Animals were anesthetized for ETO surgery, ear examinations with middle ear fluid sampling and auditory testing. Anesthesia for surgery and hearing testing was induced with intramuscular ketamine, 50 mg/kg, and xylazine, 5 mg/kg. Animals were anesthetized for ear examinations and middle ear fluid sampling by inhaled isoflurane. Animals were placed in an anesthetic chamber with isoflurane and oxygen until response to toe pinch was abolished. Anesthesia was maintained with the animals breathing isoflurane and oxygen by nose mask.

Assessment of Middle Ear Inflammation: Otomicroscopy was performed weekly before and after middle ear aspiration and reinjection. Middle ear fluid samples were cultured on chocolate agar for 18-24 hours in 10% CO₂ at 37° C. Speciation was not routinely performed. Any ears demonstrating inflammation of the tympanic membrane (opacification or erythema) with bacterial growth on 2 serial middle ear fluid cultures were deemed to have otitis media.

Temporal Bone Histopathology: After the final audiometric assessment, animals were euthanized. Temporal bones were removed from 2 animals in each group, fixed in 10% buffered formalin, and processed as described by Schuknecht. Specimens were embedded in celloidin and horizontally sectioned at 20 micrometers from superior to inferior. Every tenth section was stained with hematoxylin and eosin and examined microscopically.

Statistical Analysis: The primary outcome measures were the electrocochleographic thresholds. Ears with auditory measurements beyond the limits of instrument detection (>100 db) had thresholds were given a value of 118 db. These censored values were treated as measured values for the purposes of the statistical analysis. Data quality was investigated using diagnostic plots representing the difference between the thresholds before and thresholds after treatment.

The Multivariate Analysis of Variance (MANOVA) was used to test for significant differences among the treatment groups. The groups were defined for all measurements (clicks and tones) for the five factor levels (i.e., different treatment groups). The test was done at the multivariate level to detect global differences for all measurements. If this difference did not occur for all measurements, an ANOVA was not performed. A multivariate t-test was used to test a clinical drop in hearing (0 db).

### Results

The experiment began with 96 animals.

Persistence of middle ear effusion was demonstrated by a meniscus on otomicroscopy, type B or C tympanograms, or recovery of fluid on middle ear sampling. Persistence of middle ear effusion at the final treatment day was observed in 88% of saline-injected ears, 96% of vehicle ears, 100% of α1-antitrypsin ears, 83% of ilomastat ears, and 94% of combined α1-antitrypsin and ilomastat ears. These differences were not significant.

All treatment groups demonstrated a statistically insignificant average loss in long-term hearing (0 db) for all clicks and tones (p=0.20 for alpha 1-antitrypsin; p=0.15 for the combination of alpha 1-antitrypsin and ilomastat; p=0.29 for ilomastat; p=0.21 for saline, and p=0.71 for vehicle). In addition, hearing in all treatment groups was statistically insignificantly different from one another (p=0.5625).

Our observations suggest that these protease inhibitors are not toxic, even when chronically applied to non-inflamed middle ears of chinchillas.

The chinchilla has been widely used by researchers for studies of ototoxicity. The non-infected chinchilla inner ear is exquisitely sensitive to the application of a variety of agents to the middle ear, such as acetic acid and other ototopical preparations that have commonly been used to treat chronic suppurative otitis in humans. The non-inflamed chinchilla ear tends to bias toward an ototoxic effect. We induced bilateral Eustachian tube dysfunction to slow the middle ear clearance of the protease inhibitors, and minimal, if any, inflammation was observed in these animals' ears. Hence, the stability of hearing in chinchillas after 4 weeks of exposure to alpha 1-antitrypsin and/or ilomastat suggests strongly that these agents are likely to be safe in humans.

It is understood that the examples and embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes to the same extent as if each individual publication, patent or patent application were specifically and individually indicated to be so incorporated by reference.

What is claimed is:

1. A method of treating otitis media in a mammal comprising administering to the mammal an effective amount of alpha one-antitrypsin.

2. The method of claim 1 further comprising administering an effective amount of an antibiotic.

3. The method of claim 1 wherein the alpha one-antitrypsin is administered in a liquid.

4. The method of claim 1 wherein the alpha one-antitrypsin is administered as a dry powder.

5. The method of claim 1 further comprising administering an effective amount of a steroid.

6. The method of claim 1 wherein the mammal is a human.

7. The method of claim 1 wherein the mammal to be treated has a perforated tympanic membrane.

8. The method of claim 7 wherein the perforated tympanic membrane is due to tympanostomy.

9. The method of claim 7 or claim 8 wherein the mammal is a human.

10. The method of claim 1 wherein the otitis media is selected from the group consisting of recurrent acute otitis media (RAOM), chronic otitis media with effusion (COME), acute post-tympanostomy otorrhea (APTO), chronic suppurative otitis media (CSOM), and choleastoma.

11. The method of claim 10 wherein the type of otitis media is APTO or CSOM.

12. The method of claim 11 further comprising administering an effective amount of an antibiotic.
